(19) United States
(12) Patent Application Publication
(10) Pub. No.: US 2014/0205998 A1
(43) Pub. Date: Jul. 24, 2014

(54) METHOD FOR ENHANCING EXTRACTION EFFICIENCY OF MIRNA FROM CELLS BY THE ADDITION OF TRITON X-100

(71) Applicant: KOREA INSTITUTE OF SCIENCE AND TECHNOLOGY, Seoul (KR)

(72) Inventors: Eun Joo SONG, Seoul (KR); Eunmi BAN, Seoul (KR); Dong-Kyu CHAE, Seoul (KR)

(73) Assignee: KOREA INSTITUTE OF SCIENCE AND TECHNOLOGY, Seoul (KR)

(21) Appl. No.: 13/758,357

(22) Filed: Feb. 4, 2013

(30) Foreign Application Priority Data
Jan. 23, 2013 (KR) 10-2013-0007541

(51) Int. Cl. C12Q 1/68

(52) U.S. Cl. CPC C12Q 1/6806; C12Q 1/6813
USPC 435/6.11

(57) ABSTRACT

The present invention relates to a method for improving detection efficiency of miRNAs existing in cells in trace amounts by adding Triton X-100. In accordance with the present invention, miRNAs existing in a sample in trace amounts can be quantitatively analyzed in short time. Further, the miRNA detection method according to the present invention wherein Triton X-100 is used can improve detection efficiency by about 2 times as compared to when only a TRIzol reagent is used.

### CROSS-REFERENCE TO RELATED APPLICATIONS

This application claims priority under 35 U.S.C. §119 to Korean Patent Application No. 10-2013-0007541, filed on Jan. 23, 2013, in the Korean Intellectual Property Office, the disclosure of which is incorporated herein by reference in its entirety.

### BACKGROUND

(a) Technical Field

The present invention relates to a method for detecting miRNAs existing in a sample in trace amounts and a kit for detecting the same.

(b) Background Art

A microRNA (miRNA) is a very short non-coding RNA consisting of 21-22 nucleotides on average. By regulating other genes through inhibition of translation of mRNA, it controls cellular differentiation, embryogenesis, metabolism and oncogenesis. It is thought that about 30% of the total genes of the human genome are regulated by miRNAs. The miRNAs are generated through transcription of individual genes in the non-coding regions. The miRNA is transcribed from a pri-miRNA which is a precursor transcribed in the nucleus by RNA polymerase II. The pri-miRNA is cleaved by the RNase III enzyme called Drosha (dsRNA-specific ribonuclease) to produce a pre-miRNA having a hairpin loop structure. The hairpin loop of the pre-miRNA is exported out of the nucleus by the proteins exportin-5 and Ran-GTP, which serve as cofactors, and processed into a miRNA duplex about 22 nucleotides in length by the action of the RNase III enzyme Dicer and TRBP (transactivation-responsive RNA binding protein). The miRNA duplex binds with RISC (RNA-induced silencing complex) and regulates genes by cleaving mRNAs or preventing translation.

Various kinds of miRNAs and target genes regulated thereby may be useful in predicting the mechanisms of various diseases. Since abnormally increased or decreased miRNA expression is observed in various diseases such as cancer, diabetes and cardiovascular diseases, the miRNA is recognized as a biomarker for diagnosing and predicting of diseases.

The miRNAs exist in trace amounts in biological substances. Accordingly, a selective and highly sensitive analysis method as well as an adequate extraction method is necessary to detect them. At present, the reverse transcription polymerase chain reaction technique is mainly used for miRNA detection. Although the reverse transcription polymerase chain reaction technique is an excellent quantitative analysis method, it is costly, is problematic in reproducibility and requires a very long analysis time of 3-4 hours or longer because amplification is necessary. Therefore, other analysis methods are studied in addition to the reverse transcription polymerase chain reaction technique. Among them, capillary electrophoresis is studied a lot since it exhibits excellent quantitativity, reproducibility and resolution and, above all, short analysis time of less than 1 hour. However, since this method does not include an amplification process, development of an extraction method providing maximized extraction efficiency is required to detect the miRNAs existing in trace amounts.

Throughout the specification, a number of publications and patent documents are referred to and cited. The disclosure of the cited publications and patent documents is incorporated herein by reference in its entirety to more clearly describe the state of the related art and the present invention.

### SUMMARY

The inventors of the present invention have made efforts to establish an analysis method allowing detection of miRNAs existing in cells in trace amounts with high sensitivity in short time. As a result, they have found that trace amounts of miRNAs can be detected with high sensitivity when Triton X-100 is added to an extraction method using TRIzol.

The present invention is directed to providing a method for improving detection efficiency of miRNAs existing in cells in trace amounts by adding Triton X-100.

The present invention is also directed to providing a kit for detecting miRNAs.

Other features and aspects of the present invention will be apparent from the following detailed description, drawings and claims.

### BRIEF DESCRIPTION OF THE DRAWINGS

The above and other objects, features and advantages of the present invention will now be described in detail with reference to certain exemplary embodiments thereof illustrated in the accompanying drawings which are given hereinbelow by way of illustration only, and thus are not limitative of the invention, and wherein:

FIG. 1 shows a process for extracting miRNAs from cells for quantitative or qualitative analysis of miRNAs;

FIG. 2 shows that, when Triton X-100 is added to a miRNA extraction method using TRIzol reagent, the peak intensity of a DNA-miRNA complex resulting from hybridization with a single-stranded DNA having a fluorescent residue increases, the increase varying with the concentration of Triton X-100 and giving the highest intensity when 2% Triton X-100 is used;

FIG. 3 shows that, when 2% Triton X-100 is added to a miRNA extraction method using TRIzol reagent, the peak intensity of a DNA-miRNA complex resulting from hybridization with a single-stranded DNA having a fluorescent residue changes with incubation time and the highest intensity is achieved when incubation is performed at room temperature for 20 minutes; and

FIGS. 4a and 4b show a result of hybridizing total RNAs extracted from H9c2 cardiomyocytes with or without adding Triton X-100 at the concentration giving the highest intensity with single-stranded DNAs having fluorescent residues specific for miRNA-21 and detecting by CE/LIF (The amount of miRNA-21 extracted from the H9c2 cardiomyocytes is compared from the peaks of unreacted DNA and DNA-miRNA-21 complex).

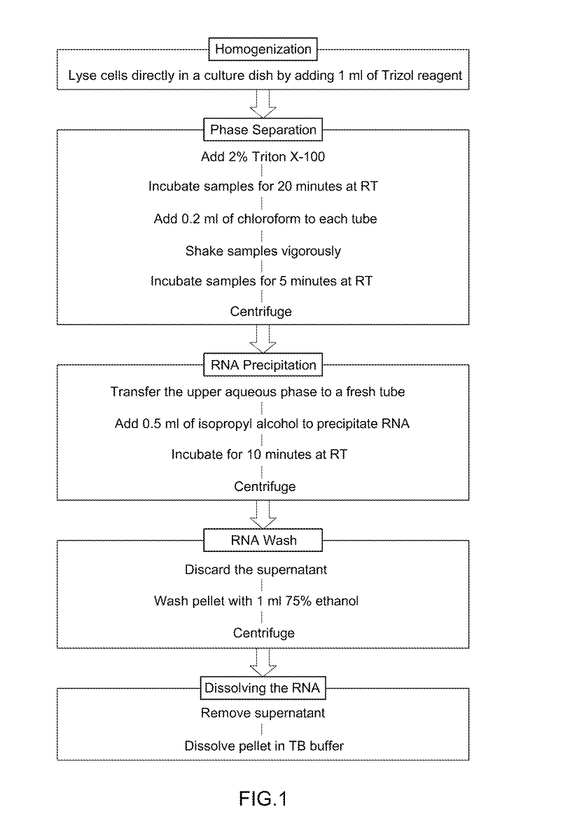

### DETAILED DESCRIPTION

Hereinafter, reference will now be made in detail to various embodiments of the present invention, examples of which are illustrated in the accompanying drawings and described below. While the invention will be described in conjunction with exemplary embodiments, it will be understood that the present description is not intended to limit the invention to those exemplary embodiments. On the contrary, the invention is intended to cover not only the exemplary embodiments, but also various alternatives, modifications, equivalents and other embodiments, which may be included within the spirit and scope of the invention as defined by the appended claims.

In an aspect, the present invention provides a method for improving detection efficiency of microRNAs (miRNAs) existing in cells in trace amounts by adding Triton X-100 (polyethylene glycol p-(1,1,3,3-tetramethylbutyl)-phenyl ether), including:

(a) lysing cells using a cell lysis buffer containing a TRIzol reagent;

(b) adding Triton X-100 and incubating the cells;

(c) obtaining a cell extract through centrifugation; and

(d) detecting miRNAs from the cell extract.

The inventors of the present invention have made efforts to establish an analysis method allowing detection of miRNAs existing in cells in trace amounts with high sensitivity in short time. As a result, they have found that trace amounts of miRNAs can be detected with high sensitivity when Triton X-100 is added to an extraction method using TRIzol.

The miRNA may have an important role in predicting various diseases. That is to say, since abnormally increased or decreased miRNA expression is observed in various diseases such as cancer, diabetes and cardiovascular diseases, the miRNA may be used as a biomarker for diagnosing and predicting of diseases. However, since the miRNA exists in trace amounts in the body, a selective and highly sensitive analysis method is necessary for detection. At present, microarray techniques and reverse transcription polymerase chain reaction techniques are frequently used for miRNA detection. Although the microarray technique allows detection of many kinds of miRNAs, quantitativity is very poor. The reverse transcription polymerase chain reaction technique is problematic in that it is costly, has reproducibility problem and, above all, requires a very long analysis time of 3-4 hours or longer.

For extraction of RNAs, a method using TRIzol is commonly employed. However, this method is limited in extraction of miRNAs since they exist in trace amounts.

As used herein, the term “trace amount” refers to an amount in the level of femtomolar (fM) or less. Specifically, the “trace amount” may refer to an amount of 500 fM or less, more specifically 100 fM or less, most specifically 50 fM or less.

The detection method developed by the inventors of the present invention allows detection of miRNAs existing in cells in trace amounts in short time, specifically within 1 hour.

The steps of the method of the present invention will be described in detail.

(a): Lysis of Cells Using Cell Lysis Buffer Containing TRIzol Reagent

In the first step, cells from which miRNAs will be extracted are lysed. In this step, any buffer for lysing cells may be used. Specifically, a cell lysis buffer containing a TRIzol reagent is used.

The TRIzol reagent is a reagent widely known in the art used in RNA/DNA/protein extraction and is composed of phenol, guanidinium thiocyanate, etc. The amount of the TRIzol reagent used in the present invention may vary depending on purposes. It may be included in an amount of specifically 0.01-100 mL, more specifically 0.1-10 mL.

(b): Addition of Triton X-100 and Incubation

This step is one of the features of the present invention. If Triton X-100 is added to a miRNA extraction method using TRIzol only, the miRNAs existing in cells in trace amounts may be extracted in an amount of specifically 1.2-4 times, more specifically 1.5-3 times, larger as compared to when only TRIzol is used.

The Triton X-100 used in the present invention is a kind of a nonionic surfactant and is also called polyethylene glycol p-(1,1,3,3-tetramethylbutyl)-phenyl ether.

In the present invention, the Triton X-100 may be added at a concentration of specifically 0.5-10%, more specifically 1-5%, most specifically 1.5-3%. If the concentration of the Triton X-100 is below 0.5% or above 10%, the extraction amount of miRNAs may not increase significantly.

For increase of the miRNA extraction amount, incubation may be carried out after adding the Triton X-100. The incubation may be carried out specifically for 1-100 minutes, more specifically for 5-30 minutes, most specifically for 15-30 minutes. If the incubation time is shorter than 1 minute or longer than 100 minutes, the extraction amount of miRNAs may not increase significantly.

(c): Obtainment of Cell Extract Through Centrifugation

After the incubation is completed, a cell extract is obtained. A high-speed or low-speed centrifuge may be used to obtain the cell extract. Those skilled in the art may select an appropriate centrifuge and centrifugation time to suit the purpose. When a Gyrozen centrifuge is used, the centrifugation may be performed at 1,000-30,000 RCF, specifically at 10,000-15,000 RCF, for 5-30 minutes, specifically for 10 minutes-20 minutes.

(d): Detection of miRNAs from Cell Extract

Finally, miRNAs are detected from the cell extract.

Since the effective miRNA detection method of the present invention wherein Triton X-100 is used exhibits a miRNA extraction amount of about 2 times or larger than the method wherein Triton X-100 is not added to the TRIzol reagent, it may be employed to effectively detect miRNAs utilizing various miRNA detection methods known in the art.

In a specific embodiment of the present invention, the detection method of the present invention may detect miRNAs existing in cells in trace amounts in short time, specifically within 1 hour.

In a specific embodiment of the present invention, the step (d) may include: (d-1) extracting RNAs from the cell extract; (d-2) hybridizing the extracted RNAs with free DNAs to which fluorescent materials specific for the miRNAs expected to exist in the cell extract are bound; (d-3) separating and detecting DNA-miRNA complexes using a capillary electrophoresis (CE)/laser induced fluorescence (LIF) system equipped with an LIF detector; and (d-4) identifying the presence of miRNAs existing in cells in trace amounts by identifying the peaks of the DNA-miRNA complexes.

The detection method of the present invention may be used for diagnosis of various diseases and prediction of prognosis thereof. Specifically, it may be used for diagnosis of cardiovascular diseases including myocardial infarction.

Various miRNAs are known as biomarkers of cardiovascular diseases. When the detection method of the present invention is used for detection of the biomarkers of cardiovascular diseases, the miRNA may be specifically miRNA-1, miRNA-21, miRNA-133, miRNA-208 or miRNA-499, more specifically miRNA-21 or miRNA-499, most specifically SEQ ID NO 1.

A labeled fluorescent material may provide a signal allowing detection of the hybridization. The label may be attached to an oligonucleotide. The label that can be used in the present invention may be various fluorescent materials including fluorescein, phycoerythrin, rhodamine, lissamine, Cy3 and Cy5 (Pharmacia), and the like. Specifically, 6-carboxyfluorescein may be used. The labeling may be performed according to the methods commonly employed in the art. For example, nick translation, random priming (Multiprime DNA labeling systems booklet, Amersham (1989)) or kination (Maxam & Gilbert, Methods in Enzymology, 65: 499 (1986)) method may be used.

An optimal hybridization condition may be determined by referring to Joseph Sambrook, et al., Molecular Cloning, A Laboratory Manual, Cold Spring Harbor Laboratory Press, Cold Spring Harbor, N.Y. (2001) and Haymes, B. D., et al., Nucleic Acid Hybridization, A Practical Approach, IRL Press, Washington, D.C. (1985). A stringent condition for hybridization may be determined by adjusting temperature, ionic strength (buffer concentration) and the presence of other compounds such as organic solvents. The stringent condition may be determined differently depending on the hybridized sequences. Various kinds of hybridization buffers commonly used in the related art may be used in the present invention. A buffer exhibiting the highest hybridization efficiency may be selected. For example, TNM buffer, PBS buffer, Tris-Cl buffer, SSC buffer, HEN buffer or TEN buffer may be used as the hybridization buffer. Specifically, TEN buffer may be used.

In a specific embodiment of the present invention, the CE/LIF system may be equipped with a laser-induced fluorescence (LIF) detector.

When the CE/LIF system is used, the hybridized DNA-miRNA complex may be separated using an uncoated capillary. Specifically, the capillary may have an inner diameter of 50-100 μm and a length of 20-60 cm, but is not limited thereto. The hybridized DNA-miRNA complex may be separated by applying a voltage of 10-20 kV, specifically 16 kV, into the capillary using Tris-borate buffer as a separation buffer.

The wavelength of the LIF detector may be different depending on the fluorescent material to be detected. When 6-carboxyfluorescein is used as the fluorescent material, the excitation wavelength may be specifically 400-500 nm, most specifically 488 nm, and the emission wavelength may be specifically 500-600 nm, most specifically 520 nm.

In another aspect, the present invention provides a kit for detecting miRNAs.

Since the kit of the present invention is for detecting the miRNAs described above, description about the matters described above will be omitted to avoid unnecessary redundancy.

In a specific embodiment of the present invention, the present invention provides a kit for detecting miRNAs existing in trace amounts of not greater than 50 femtomolar, including: (a) a TRIzol reagent; (b) Triton X-100; (c) fluorescent materials that can be specifically hybridized with the miRNAs; (d) a hybridization buffer; and (e) a buffer for separation of a DNA-miRNA complexes

In a specific embodiment of the present invention, the miRNA may be miRNA-21.

In a specific embodiment of the present invention, the hybridization buffer may be one or more selected from a group consisting of TNM buffer, PBS buffer, Tris-Cl buffer, SSC buffer, HEN buffer and TEN buffer.

In a specific embodiment of the present invention, the separation buffer may be a Tris-borate buffer.

In a specific embodiment of the present invention, the fluorescent material may be 6-carboxyfluorescein.

### EXAMPLES

The present invention will be described in more detail through examples. The following examples are for illustrative purposes only and it will be apparent to those skilled in the art not that the scope of this invention is not limited by the examples.

### Example 1

### Establishment of Optimal Concentration of Triton X-100

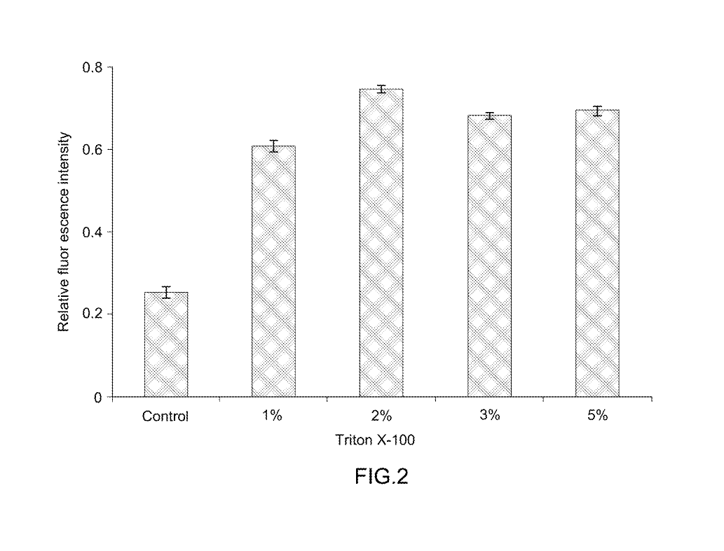

Total RNAs were extracted from H9c2 cardiomyocytes by adding Triton X-100 of different concentrations to a TRIzol reagent commonly used for extraction of RNAs or miRNAs. Free DNAs labeled with the fluorescent material 5′-carboxyfluorescein phosphoramidite (6-FAM), which is specific for miRNA-21 (5′-UAGCUUAUCAGACUGAUGUUGA-3′), were denatured at 95° C. for 5 minutes, hybridized with the extracted total RNAs at 40° C. for 15 minutes in TEN buffer (50 mM Tris-Ac (pH 8.0), 10 mM EDTA, 50 mM NaCl) and analyzed using a capillary electrophoresis (CE) system equipped with a laser-induced fluorescence (LIF) detector. The CE system was PA 800 plus CE system (Beckman Coulter, Fullerton, Calif., USA) and the LIF detector was Beckman P/ACE System Laser Module 488 with excitation and emission wavelengths of 488 nm and 520 nm, respectively. DNA-miRNA-21 complexes were separated in 100 mM Tris-borate buffer (pH 10.0) by applying a voltage of 16 kV into an uncoated capillary (Beckman Coulter) having an inner diameter of 75 μm and a length of 30 cm. Sample injection was carried out at 0.5 psi for 5 seconds. As seen from FIG. 2, the peak intensity of the DNA-miRNA-21 complex changed significantly with the concentration of Triton X-100. The highest peak intensity was achieved when the concentration of Triton X-100 was 2%.

### Example 2

### Establishment of Optimal Incubation Time

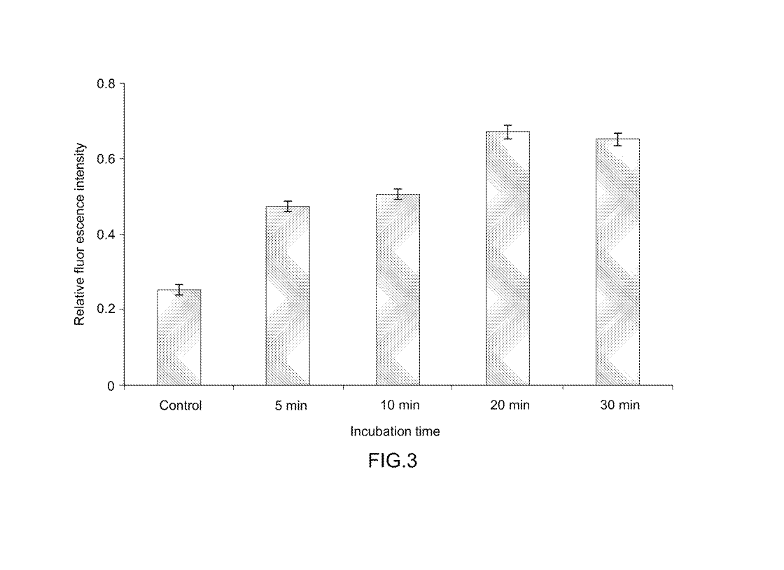

Total RNAs were extracted from H9c2 cardiomyocytes by adding 2% Triton X-100 to a TRIzol reagent commonly used for extraction of RNAs or miRNAs while varying incubation time (FIG. 3). Analysis was carried out under the same condition as Example 2. It was confirmed that the peak intensity of the DNA-miRNA-21 complex changed with the incubation time. When 2% Triton X-100 was used, the highest peak intensity was achieved when the incubation time was 20 minutes.

### Example 3

### Detection of miRNAs from Cardiomyocytes with or without Triton X-100

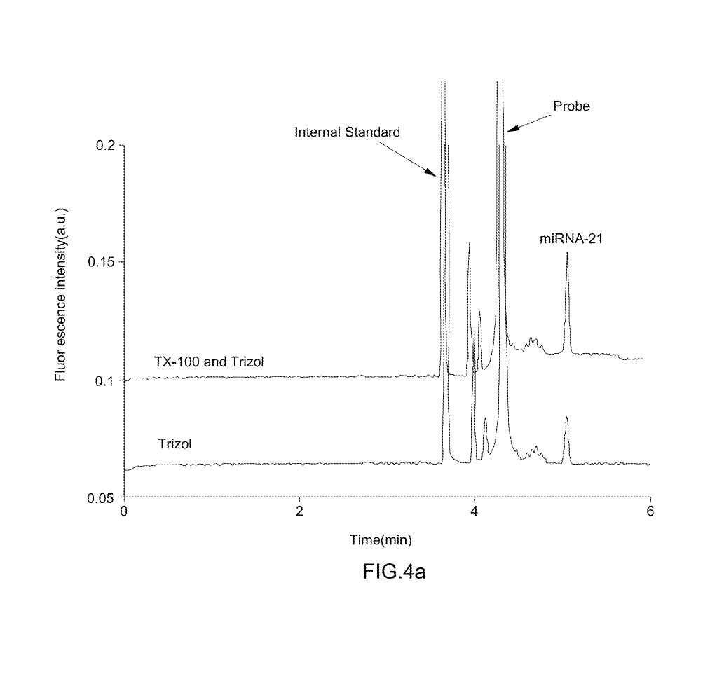

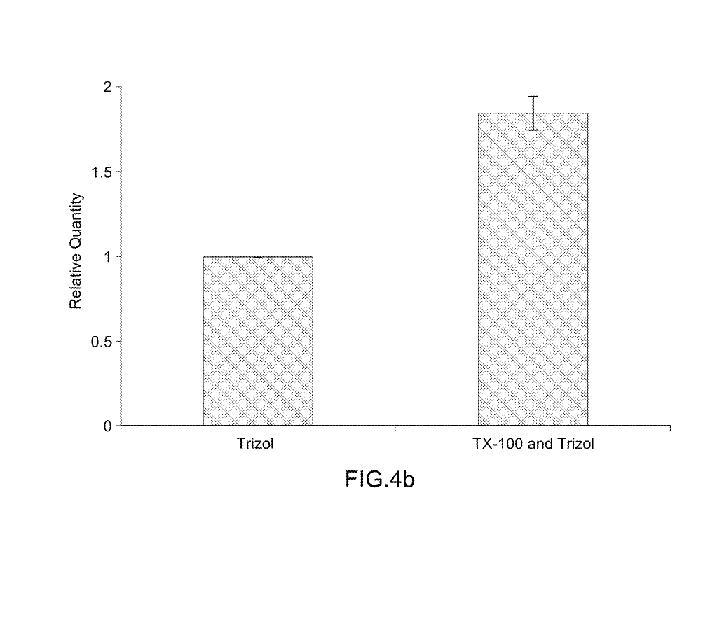

Cardiomyocytes were cultured in DMEM containing 10% FBS and 1 vol % penicillin-streptomycin. The medium was replaced every other day and the incubator used to culture the cells was maintained at 37° C. and 5% CO₂. Total RNAs were extracted from 1×10⁶ cells on a 100 mm dish using TRIzol with or without 2% Triton X-100 added, with an incubation time of 20 minutes. DNAs labeled with the fluorescent material 6-FAM, which is specific for miRNA-21, were denatured at 95° C. for 5 minutes, hybridized with the extracted total RNAs at 40° C. for 15 minutes in TEN hybridization buffer and the resulting complexes were analyzed using a CE system equipped with an LIF detector. The CE system was PA 800 plus CE system (Beckman Coulter, Fullerton, Calif., USA) and the LIF detector was Beckman P/ACE System Laser Module 488 with excitation and emission wavelengths of 488 nm and 520 nm. The DNA-miRNA-21 complexes were separated in 200 mM Tris-borate buffer (pH 10.0) by applying a voltage of 16 kV into an uncoated capillary (Beckman Coulter) having an inner diameter of 75 μm and a length of 30 cm. Sample injection was carried out at 0.5 psi for 5 seconds. The result is shown in FIGS. 4a and 4b. The peak of the DNA-miRNA complex was observed from both cells with or without Triton X-100 added, but the intensity of the DNA-miRNA complex peak was about 2 times stronger in the cells to which Triton X-100 was added.

The features and advantages of the present disclosure may be summarized as follows:

(i) The present invention provides a method for improving detection efficiency of miRNAs existing in cells in trace amounts by adding Triton X-100.

(ii) The present invention allows quantitative analysis of miRNAs existing in a sample in trace amounts in short time.

(iii) The miRNA detection method according to the present invention wherein Triton X-100 is used can improve detection efficiency by about 2 times as compared to when only a TRIzol reagent is used.

The present invention has been described in detail with reference to specific embodiments thereof. However, it will be appreciated by those skilled in the art that various changes and modifications may be made in these embodiments without departing from the principles and spirit of the invention, the scope of which is defined in the appended claims and their equivalents.

What is claimed is:

1. A method for improving detection efficiency of miRNAs existing in cells in trace amounts by adding Triton X-100 (polyethylene glycol p-(1,1,3,3-tetramethylbutyl)-phenyl ether), comprising:
lysing cells using a cell lysis buffer containing a TRIzol reagent;
adding Triton X-100 and incubating the cells;
obtaining a cell extract through centrifugation; and
detecting miRNAs from the cell extract.

2. The method according to claim 1, wherein the concentration of the Triton X-100 is 1-5%.

3. The method according to claim 1, wherein said incubation is carried out for 5-30 minutes.

4. The method according to claim 1, wherein said detecting miRNAs from the cell extract comprises:
extracting RNAs from the cell extract;
hybridizing the extracted RNAs with free DNAs to which fluorescent materials specific for the miRNAs expected to exist in the cell extract are bound;
separating and detecting DNA-miRNA complexes using a capillary electrophoresis (CE)/laser induced fluorescence (LIF) system equipped with an LIF detector; and
identifying the presence of miRNAs existing in cells in trace amounts by identifying the peaks of the DNA-miRNA complexes.

5. The method according to claim 4, wherein the LIF detector has an excitation wavelength of 400-500 nm and an emission wavelength of 500-600 nm.

6. The method according to claim 4, wherein said separation of the DNA-miRNA complexes is performed using an uncoated capillary.

7. The method according to claim 6, wherein the uncoated capillary has an inner diameter of 50-100 μm and a length of 20-60 cm.

8. The method according to claim 6, wherein said separation is performed by applying a voltage of 10-20 kV into the uncoated capillary using Tris-borate buffer.

9. The method according to claim 1, wherein the cells are cardiomyocytes.

10. The method according to claim 1, wherein the miRNA is miRNA-21.

11. The method according to claim 10, wherein the miRNA-21 is miRNA of SEQ ID NO 1.

12. The method according to claim 4, wherein fluorescent material is 6-carboxyfluorescein.

13. The method according to claim 4, wherein said hybridization is performed using one or more hybridization buffer selected from a group consisting of TNM buffer, PBS buffer, Tris-Cl buffer, SSC buffer, HEN buffer and TEN buffer.

14. The method according to claim 13, wherein the hybridization buffer is TEN buffer.

15. A kit for detecting miRNAs existing in trace amounts of not greater than 50 femtomolar, which is for use in a capillary electrophoresis/laser-induced fluorescence (CE/LIF) system, comprising: a TRIzol reagent; Triton X-100; fluorescent materials that can be specifically hybridized with the miRNAs; a hybridization buffer; and a buffer for separation of DNA-miRNA complexes.

16. The kit for detecting miRNAs according to claim 15, wherein the miRNA is miRNA-21.

17. The kit for detecting miRNAs according to claim 15, wherein the hybridization buffer is one or more selected from a group consisting of TNM buffer, PBS buffer, Tris-Cl buffer, SSC buffer, HEN buffer and TEN buffer.

18. The kit for detecting miRNAs according to claim 15, wherein the buffer for separation is a Tris-borate buffer.

19. The kit for detecting miRNAs according to claim 15, wherein the fluorescent material is 6-carboxyfluorescein.


SEQUENCE LISTING (1 sequence)

SEQ ID NO: 1 - RNA, 22 nt, Artificial Sequence
  miRNA-21
uagcuuaucagacugauguuga